(19) United States
(12) Patent Application Publication
(10) Pub. No.: US 2016/0129143 A1
(43) Pub. Date: May 12, 2016

(54) Odor Elimination System

(71) Applicant: Leisa Lingwall, Sandy, UT (US)

(72) Inventor: Leisa Lingwall, Sandy, UT (US)

(21) Appl. No.: 14/928,627

(22) Filed: Oct. 30, 2015

Related U.S. Application Data
(60) Provisional application No. 62/078,349, filed on Nov. 11, 2014.

(51) Int. Cl. A61L 2/18; B05B 1/14; B05B 11/02

(52) U.S. Cl. CPC A61L 2/18; B05B 11/02; B05B 1/14

(57) ABSTRACT

A odor elimination system includes a pad that may be positioned on a support surface and a carpet is positioned on the pad. An injection unit is provided and the injection unit contains a fluid odor eliminator. The injection unit includes a plurality of needles. The needles penetrate the carpet and the pad. Thus, the injection unit may deliver the fluid odor eliminator into the pad thereby facilitating the fluid odor eliminator to eliminate an odor associated with a contaminant in the carpet and the pad.

### CROSS REFERENCE TO RELATED APPLICATIONS

This application claims the benefit of the provisional application Ser. No. 62/078,349 filed on Nov. 11, 2014.

### BACKGROUND OF THE DISCLOSURE

### Field of the Disclosure

The disclosure relates to elimination devices and more particularly pertains to a new elimination device for eliminating an odor associated with a contaminant in a carpet.

### SUMMARY OF THE DISCLOSURE

An embodiment of the disclosure meets the needs presented above by generally comprising a pad that may be positioned on a support surface and a carpet is positioned on the pad. An injection unit is provided and the injection unit contains a fluid odor eliminator. The injection unit includes a plurality of needles. The needles penetrate the carpet and the pad. Thus, the injection unit may deliver the fluid odor eliminator into the pad thereby facilitating the fluid odor eliminator to eliminate an odor associated with a contaminant in the carpet and the pad.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a top perspective view of a odor elimination system according to an embodiment of the disclosure.

FIG. 2 is a left side view of an embodiment of the disclosure.

FIG. 3 is a cross sectional view taken along line 3-3 of FIG. 2 of an embodiment of the disclosure.

FIG. 4 is a left side view of an alternative embodiment of the disclosure.

FIG. 5 is a front view of an alternative embodiment of the disclosure.

### DESCRIPTION OF THE PREFERRED EMBODIMENT

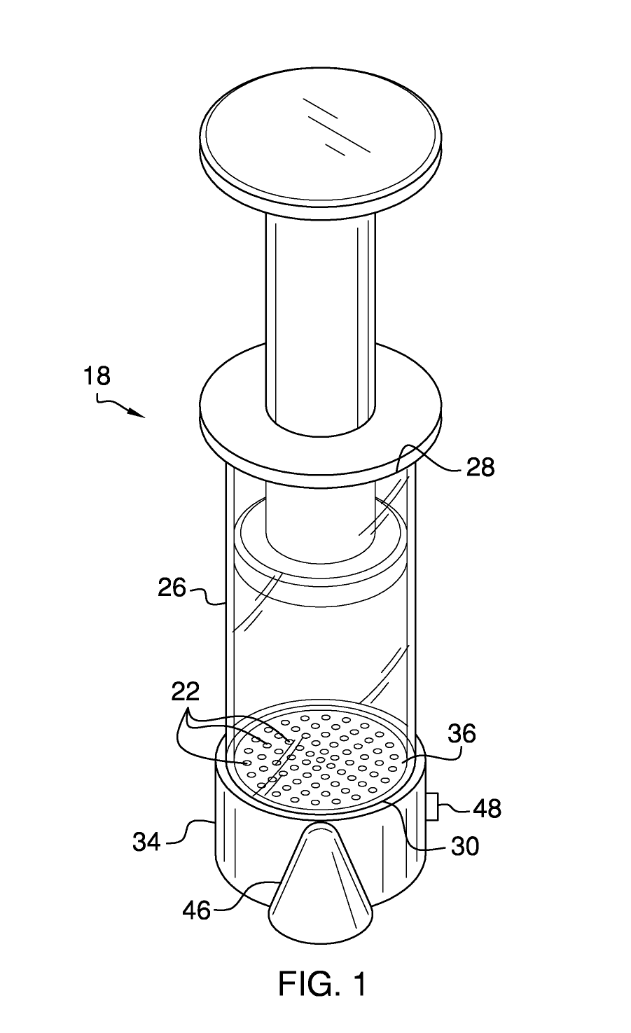

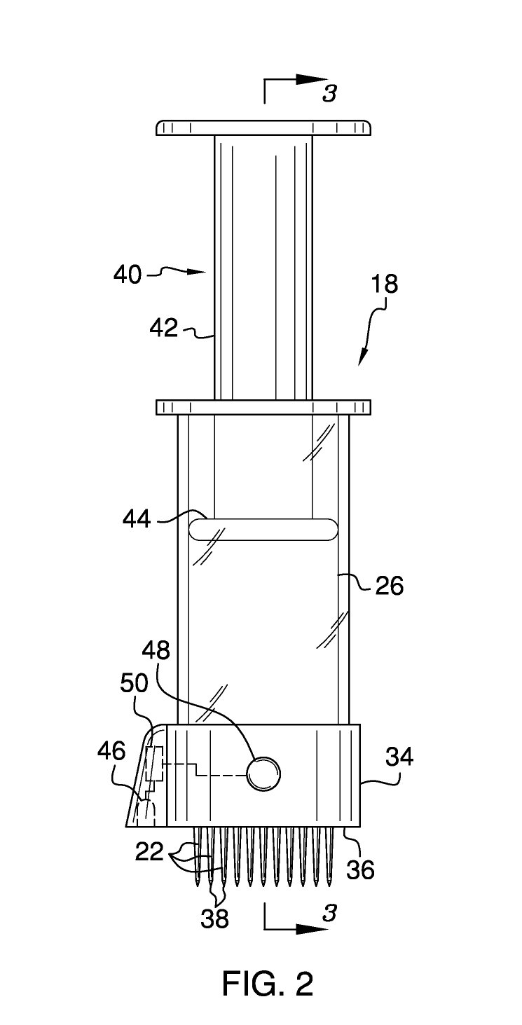

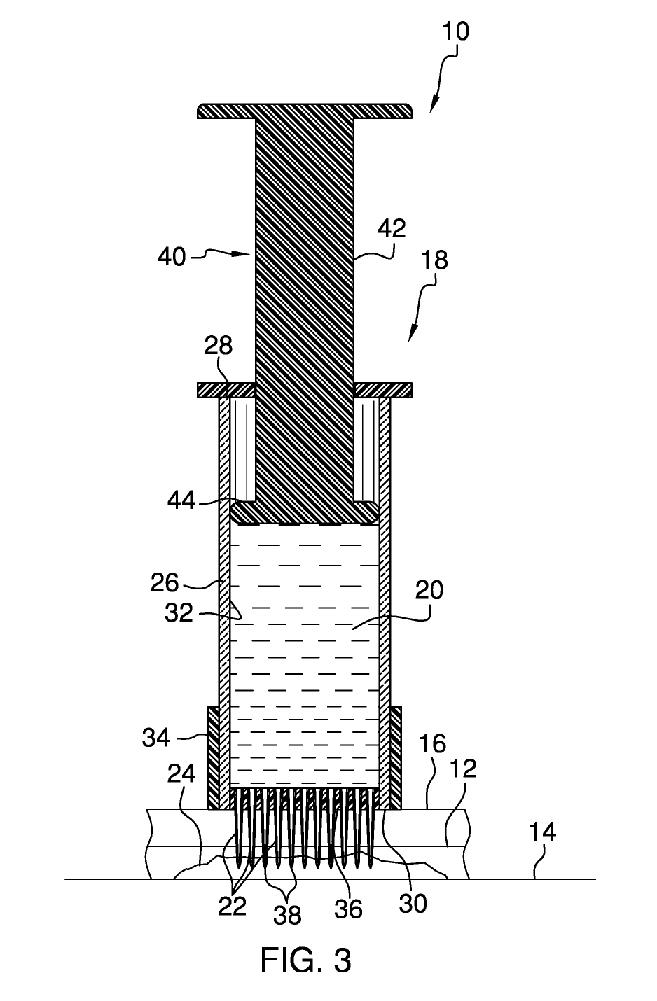

With reference now to the drawings, and in particular to FIGS. 1 through 5 thereof, a new elimination device embodying the principles and concepts of an embodiment of the disclosure and generally designated by the reference numeral 10 will be described.

As best illustrated in FIGS. 1 through 5, the odor elimination system 10 generally comprises a pad 12 that may be positioned on a support surface 14. A carpet 16 is positioned on the pad. The support surface 14 may be a floor or the like. The carpet 16 may be any carpet associated with residential and commercial construction. The pad 12 may be any carpet pad associated with residential and commercial construction.

An injection unit 18 is provided and the injection unit 18 may be manipulated. The injection unit 18 contains a fluid odor eliminator 20. The fluid odor eliminator 20 may comprise an odor eliminator associated with cleaning urine. The injection unit 18 includes a plurality of needles 22 and each of the needles 22 selectively penetrates the carpet 16 and the pad 12. The injection unit 18 delivers the fluid odor eliminator 20 into the pad 12. Thus, the fluid odor eliminator 20 eliminates an odor associated with a contaminant 24 in the carpet 16 and the pad 12. The contaminant 24 may comprise urine.

The injection unit 18 comprises a cylinder 26 that has a top end 28 and a bottom end 30. Each of the top end 28 and the bottom end 30 are open and the cylinder 26 is substantially hollow. The cylinder 26 contains the fluid odor eliminator 20 and the cylinder 26 has an inside surface 32.

A cup 34 is positioned around the cylinder 26 and the cup 34 is positioned adjacent to the bottom end 30. The cup 34 has a basal wall 36 and the basal wall 36 closes the bottom end 30. Each of the needles 22 extends through the basal wall 36. Each of the needles 22 is substantially hollow and each of the needles 22 is in fluid communication with an interior of the cylinder 26.

The needles 22 are spaced apart from each other and distributed on the basal wall 36. Each of the needles 22 has a distal end 38 with respect to the basal wall 36. The distal end 38 of each of the needles 22 is open and the distal end 38 of each of the needles 22 releases the fluid odor eliminator 20. Each of the needles 22 selectively penetrates the carpet 16 and the pad 12 and the distal end 38 of each of the needles 22 is positioned within the pad 12. Thus, each of the needles 22 may deliver the fluid odor eliminator 20 into the pad 12.

A plunger 40 is slidably positioned within the cylinder 26. The plunger 40 includes a shaft 42 and a plate 44. The plate 44 engages the inside surface 32 of the cylinder 26 and the plate 44 forms a fluid impermeable seal with the inside surface 32. The shaft 42 is urgeable downwardly in the cylinder 26. Thus, the plate 44 may urge the fluid odor eliminator 20 outwardly through the needles 22.

A light emitter 46 is coupled to the cup 34 and the light emitter 46 emits light onto the carpet 16. Thus, the contaminant 24 may be visually identified on the carpet 16. The light emitter 46 may comprise an LED or the like. The light emitter 46 emits ultraviolet light. Thus, the light emitter 46 facilitates the contaminant 24 to glow thereby enhancing visibility of the contaminant 24.

A switch 48 is coupled to the cylinder 26 and the switch 48 may be manipulated. The switch 48 is electrically coupled to the light emitter 46. A power supply 50 is positioned within the light emitter 46 and the power supply 50 is electrically coupled to the light emitter 46. The power supply 50 comprises at least one battery.

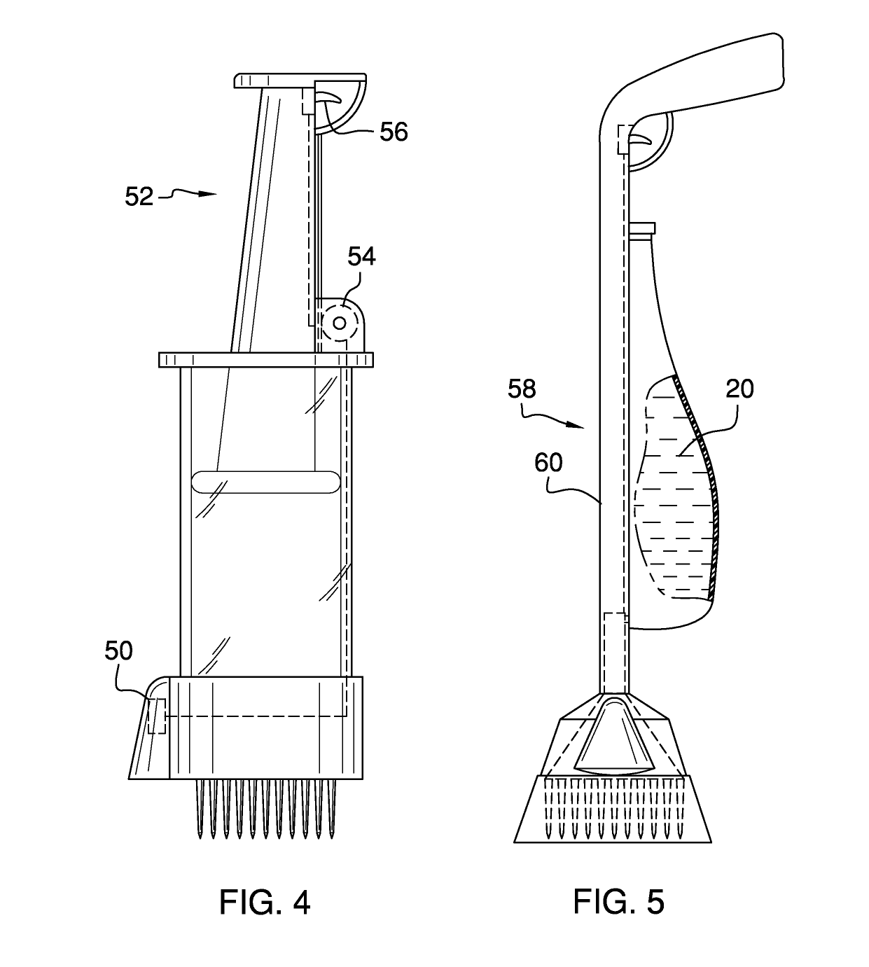

In an alternative embodiment 52 as shown in FIG. 4, a motor 54 may be coupled to the cylinder 26. The motor 54 may be mechanically coupled to the plunger 40 through any conventional mechanical means. A trigger 56 may be coupled to the cylinder 26 and the trigger 56 may be electrically coupled to the motor 54. The motor 54 may be electrically coupled the power supply 50. The motor 54 may urge the plunger 40 downwardly in the cylinder 26 when the trigger 56 is manipulated. In an alternative embodiment 58 as shown in FIG. 5, the injection unit 18 may be incorporated into a carpet cleaning machine 60 or the like.

In use, the plunger 40 is removed from the cylinder 26 and the fluid odor eliminator 20 is poured into the cylinder 26. The plunger 40 is inserted into the top end 28 of the cylinder 26. The switch 48 is manipulated and the light emitter 46 is directed to emit light onto the carpet 16 thereby facilitating visually identification of the contaminant 24. The cylinder 26 is manipulated such that the distal end 38 of each of the needles 22 penetrates the carpet 16 and the pad 12 at the contaminant's 24 location. The plunger 40 is urged downwardly in the cylinder 26 and the liquid odor eliminator 20 is distributed into the pad 12. Thus, the liquid odor eliminator 20 chemically reacts with the contaminant 24 in the pad 12 thereby eliminating the odor associated with the contaminant 24.

With respect to the above description then, it is to be realized that the optimum dimensional relationships for the parts of an embodiment enabled by the disclosure, to include variations in size, materials, shape, form, function and manner of operation, system and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An odor elimination system comprising:
a pad being configured to be positioned on a support surface;
a carpet being positioned on said pad; and
an injection unit being configured to be manipulated, said injection unit being configured to contain a fluid odor eliminator, said injection unit including a plurality of needles, said needles penetrating said carpet and said pad wherein said injection unit is configured to deliver the fluid odor eliminator into said pad thereby facilitating the fluid odor eliminator to eliminate an odor associated with a contaminant in said carpet and said pad.

2. The system according to claim 1, wherein said injection unit comprises a cylinder having a top end and a bottom end, each of said top end and said bottom end being open, said cylinder being substantially hollow wherein said cylinder is configured to contain the fluid odor eliminator, said cylinder having an inside surface.

3. The system according to claim 2, further comprising a cup being positioned around said cylinder, said cup being positioned adjacent to said bottom end, said cup having a basal wall, said basal wall closing said bottom end.

4. The system according to claim 3, wherein each of said needles is coupled to said basal wall, each of said needles being substantially hollow such that each of said needles is in fluid communication with an interior of said cylinder, said needles being spaced apart from each other and distributed on said basal wall, each of said needles having a distal end with respect to said basal wall.

5. The system according to claim 4, wherein said distal end of each of each of said needles is open wherein said distal end of each of said needles is configured to release the fluid odor eliminator, each of said needles selectively penetrating said carpet and said pad such that said distal end of each of said needles is positioned within said pad wherein each of said needles is configured to deliver the fluid odor eliminator into said pad.

6. The system according to claim 2, further comprising a plunger being slidably positioned within said cylinder, said plunger including a shaft and a plate, said plate engaging said inside surface of said cylinder, said plunger being urgeable downwardly in said cylinder wherein said plunger is configured to urge the fluid odor eliminator outwardly through said needles.

7. The system according to claim 1, further comprising:
a cup; and
a light emitter being coupled to said cup wherein said light emitter is configured to emit light onto said carpet thereby facilitating the contaminant to be visually identified on said carpet.

8. The system according to claim 7, further comprising:
a cylinder; and
a switch being coupled to said cylinder wherein said switch is configured to be manipulated, said switch being electrically coupled to said light emitter.

9. The system according to claim 8, further comprising a power supply being positioned within said light emitter, said power supply being electrically coupled to said light emitter, said power supply comprising at least one battery.

10. An odor elimination system comprising:
a pad being configured to be positioned on a support surface;
a carpet being positioned on said pad; and
an injection unit being configured to be manipulated, said injection unit being configured to contain a fluid odor eliminator, said injection unit including a plurality of needles, said needles penetrating said carpet and said pad wherein said injection unit is configured to deliver the fluid odor eliminator into said pad thereby facilitating the fluid odor eliminator to eliminate an odor associated with a contaminant in said carpet and said pad, said injection unit comprising:
a cylinder having a top end and a bottom end, each of said top end and said bottom end being open, said cylinder being substantially hollow wherein said cylinder is configured to contain the fluid odor eliminator, said cylinder having an inside surface,
a cup being positioned around said cylinder, said cup being positioned adjacent to said bottom end, said cup having a basal wall, said basal wall closing said bottom end;
each of said needles being coupled to said basal wall, each of said needles being substantially hollow such that each of said needles is in fluid communication with an interior of said cylinder, said needles being spaced apart from each other and distributed on said basal wall, each of said needles having a distal end with respect to said basal wall, said distal end of each of said needles being open wherein said distal end of each of said needles is configured to release the fluid odor eliminator, each of said needles selectively penetrating said carpet and said pad such that said distal end of each of said needles is positioned within said pad wherein each of said needles is configured to deliver the fluid odor eliminator into said pad,
a plunger being slidably positioned within said cylinder, said plunger including a shaft and a plate, said plate engaging said inside surface of said cylinder, said plunger being urgeable downwardly in said cylinder wherein said plunger is configured to urge the fluid odor eliminator outwardly through said needles,
a light emitter being coupled to said cup wherein said light emitter is configured to emit light onto said carpet thereby facilitating the contaminant to be visually identified on said carpet,
a switch being coupled to said cylinder wherein said switch is configured to be manipulated, said switch being electrically coupled to said light emitter, and
a power supply being positioned within said light emitter, said power supply being electrically coupled to said light emitter, said power supply comprising at least one battery.
